(19) United States
(12) Patent Application Publication
(10) Pub. No.: US 2020/0333309 A1
(43) Pub. Date: Oct. 22, 2020

(54) SYSTEM AND METHOD OF DETECTING FOOD ALLERGENS

(71) Applicant: AllerGuard Itd., Modiin Illit (IL)

(72) Inventors: Guy Eyal, Hod-Hasharon (IL); Shai Hershkovich, Yokneam Illit (IL)

(21) Appl. No.: 16/626,557

(22) PCT Filed: Jan. 9, 2019
(86) PCT No.: PCT/IB19/50163

§ 371 Date: Dec. 25, 2019

Related U.S. Application Data
(60) Provisional application No. 62/615,975, filed on Jan. 11, 2018.
(60) Provisional application No. 62/686,171, filed on Jun. 18, 2018.

(51) Int. Cl. G01N 33/02; G01N 33/00

(52) U.S. Cl. CPC G01N 33/02; G01N 2033/0019; G01N 33/0047

(57) ABSTRACT

A method for detecting a food allergen, the method may include (a) exposing a sensor to vapors emitted from food, wherein the sensor comprises one or more conductive paths that comprise one or more adsorbing portions that are configured to adsorb one or more volatile indicators that differ from the food allergen and are emitted from a food component that comprises the food allergen; wherein an impedance of the one or more adsorbing portions is responsive to an adsorption of at least one volatile indicator of the one or more volatile indicators; (b) measuring the one or more impedances of the one or more adsorbing portions to provide sensed information; and (c) determining a presence of the food allergen in the food, based on the sensed information.

### CROSS REFERENCE

This application claims priority from U.S. provisional patent Ser. No. 62/615,975 filing date Jan. 11, 2018, and from U.S. provisional patent Ser. No. 62/686,171 filing date Jun. 18, 2018—both are incorporated herein in their entirety.

### BACKGROUND

Food allergy is an abnormal response of the body's immune system to normally harmless proteins in the food, such as peanuts, tree nuts, seafood, milk and eggs. Whilst in most people these substances (allergens) pose no problem, in allergic individuals, the immune system identifies them as a ‘threat’ and produces an overreacted and self-perpetuating response. The signs and symptoms may range from mild to severe. They may include itchiness, swelling of the tongue, vomiting, diarrhoea, hives, laboured or shallow breathing, or low blood pressure. More severe cases may lead to anaphylaxis, acute asphyxia and even death.

Sensitivity level varies between allergy patients, and in some cases may trigger response to extremely small amounts of allergen—as low as a few micrograms of the protein in question (which would constitute several tens of micro-grams of the food substance).

### SUMMARY

There is provided a system and a method for detecting the presence of food allergens, using indicators present in the vapors coming out of the provision.

There may be provided a food allergen detector that may include: a sensor that may include one or more conductive paths, wherein the conductive paths may include one or more adsorbing portions that may be configured to adsorb one or more volatile indicators that differ from the food allergen and may be emitted from a food component that may include the food allergen; wherein the electric impedance of the one or more adsorbing portions may be responsive to adsorption of at least one volatile indicator of the one or more volatile indicators; and a measurement unit that may be configured to (a) measure, while the sensor may be exposed to vapors from the food, the one or more impedances of the one or more adsorbing portions to provide sensed information; and (b) to determine a presence of the food allergen in the food, based on the sensed information.

The measurement unit may be configured to measure an impedance of a conductive path of the one or more conductive paths by performing alternating current (AC) impedance spectroscopy.

The sensed information may represent measurements of the electric impedance of the one or more adsorbing portions at different alternating current (AC) frequencies, and wherein the measurement unit may be configured to determining the presence of the allergen by comparing the sensed information to reference information about impedances of one or more conductive paths at a presence of the one or more volatile indicators at the different AC frequencies.

The different AC frequencies may include few tens of frequencies (for example—there may be 20, 30, 40, 50, 60 or 70 different frequencies), or it may contain a frequency-domain “white noise”, where all frequencies in the given range are stimulated at once or at random.

The different AC frequencies may be within any range and may be evenly or non-evenly spaced apart from each other.

For example—the selection of few tens of frequencies (for example 71 frequencies) between 1 Hz and 50 KHz resulted in a sensitivity of below 1 part per billion. The frequencies included about fifteen frequencies below 10 Hz, fifteen frequencies between 10 and 100 Hz, five frequencies between 100 and 200 Hz, eight frequencies between 200 and 1000 Hz, fifteen frequencies between 1 KHz and 10 KHz, and eleven frequencies between 10 KHz and 50 KHz. Other allocations of frequencies may be provided. Other frequency ranges may be provided.

Yet for another example—twenty frequencies between 100 Hz and 10 KHz may be selected.

The measurement unit may be configured to determine a concentration of the allergen in the food based on the sensed information.

Each conductive path may include a pair of electrodes, and wherein an adsorbing portion of the one or more adsorbing portions electrically couples the pair of electrodes to each other, as to allow measurement of current, resistance or impedance between them.

The food allergen detector may be a mobile handheld device.

The different conductive paths of the one or more conductive paths may be tailored to sense different volatile indicators.

The food allergen detector may include one or more heating elements that may be configured to heat the one or more conductive paths while the measurement unit measures the one or more impedances of the one or more conductive paths.

The food allergen detector may include a vapors manipulator for directing the vapors towards the sensor.

The adsorbing portion belongs to a disposable portion of the mobile handheld device.

The adsorbing portion may include nanoparticles of resistance which changes in response to the adsorption of relevant molecules.

The method and detector may be applied, mutatis mutandis to detect other phenomena. Such as detection of food rotting using the same “electronic nose” to detect rotting-specific indicators.

The AC impedance measurements have found to improve the sensitivity and specificity of semiconducting quasi-molecular-imprinting gas sensing.

There may be provided a method for detecting an occurrence of an event, the method may include (a) exposing a sensor to vapors, wherein the sensor comprises one or more conductive paths that comprise one or more adsorbing portions that are configured to adsorb one or more volatile indicators that are indicative of the occurrence of the event; wherein an impedance of the one or more adsorbing portions is responsive to an adsorption of at least one volatile indicator of the one or more volatile indicators; (b) measuring the one or more impedances of the one or more adsorbing portions to provide sensed information; wherein the measuring comprises performing alternating current (AC) impedance spectroscopy; and (c) determining the occurrence of an event, based on the sensed information.

The event may or may not be meat spoilage.

There may be provided a detector that may include: a sensor that may include one or more conductive paths, wherein the conductive paths may include one or more adsorbing portions that may be configured to adsorb one or more volatile indicators that are indicative of the occurrence of the event; wherein the electric impedance of the one or more adsorbing portions may be responsive to adsorption of at least one volatile indicator of the one or more volatile indicators; and a measurement unit that may be configured to (a) measure, while the sensor may be exposed to vapors, the one or more impedances of the one or more adsorbing portions to provide sensed information; wherein the measuring includes performing alternating current (AC) impedance spectroscopy; and (b) determine the occurrence of an event, based on the sensed information.

The event may or may not be meat spoilage.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a side view of a standard dish being analyzed;

FIG. 2 is an example of a method;

FIG. 3 is an example of a method;

FIG. 4 is an example of a method;

FIG. 5 is an example of a performances of various detection methods:

FIG. 6 is an example of a food allergen detector;

FIG. 7 is an example of a food allergen detector; and

FIG. 8 is an example of a method.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a device or system capable of executing the method.

Any reference in the specification to a system or a device should be applied mutatis mutandis to a method that may be executed by the system.

Any combination of any module or unit listed in any of the figures, any part of the specification and/or any claims may be provided.

As any substance above absolute zero emits vapors (at amounts depending on temperature, of course), vapors are a good place to search for traces of any and all substances in the food dish. Naturally, vapors from organic material such as human food will contain mostly water vapor, and in much smaller quantities any volatile compounds that are present in the various substances in it. Substances which exist in very small amounts in the food dish (such as trace amounts of allergens) will contribute even smaller amounts of volatile compounds to the vapor.

There is provided a system that may include an adsorption surface or a chemical filter, that may be based on Metal-Organic-Framework, on Molecular Imprinting, or any other surface adsorption contraption to selectively-adsorb only (or almost only) the volatile compounds which will be found to point to the presence of allergens (such as sesame, peanuts, shellfish, tree nuts, or many others as specified in publications by the FDA). The contraption may be selective to a level which can create significant surface (bound) distribution of said compounds from their original (gaseous) of less than one part per million (PPM) and even less than one Parts Per Billion (PPB) level in the vapor. Such significant surface distribution can allow detection using electrochemical sensors (such as changes in resistance or impedance), or any other physical detector (such as piezoelectric sensors or optical sensors).

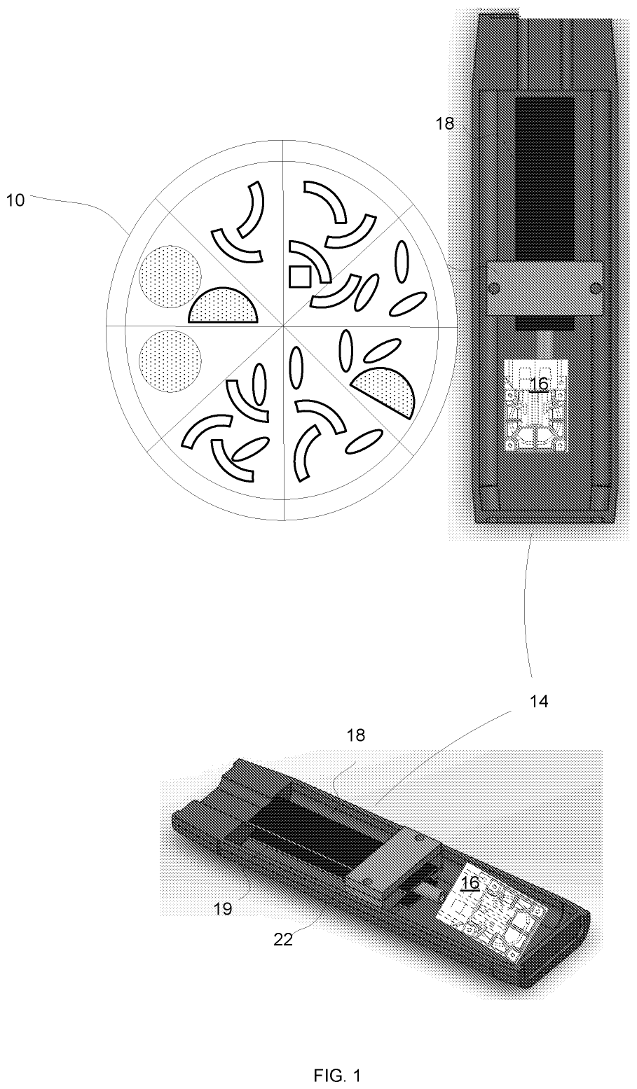

Referring now to FIG. 1 that illustrates an inspected element (such as food 10—the figure shows a pizza with various ingredients) that may be positioned on a dish (or other containing element) , being scanned by Sensor 14. Vapors coming out of the food may be taken in naturally or pumped in (as by vacuum pump 16 of sensor 14) so volatile indicators are bound to adsorbers (filters) 18 & 19 (which are selective surface adsorption contraptions). The selective adsorber may include one or more adsorbing portions (in this example two, in parallel).

Bound compounds may be then analyzed by electro-chemical resistance/impedance measurement by an ohmmeter, a potentiostat or a spectrum analyzer 22 (which may be designed as an independent unit, a Printed Circuit Board or an Integrated Circuit in a system-on-chip scheme), or by other methods such as spectrometry-scan by spectrograph (if their amount is high enough), Quartz Crystal Microbalance measurement, or any other analytical instrument. and results may be processed by a processor of the sensor 14 (integrated into PCB/IC 22) or may be sent to a processor that is not included in the sensor 14—for example the results may be sent for analysis to a specialized app on the user's smartphone (or to other processing entity), where final results are processed, determined and presented to user.

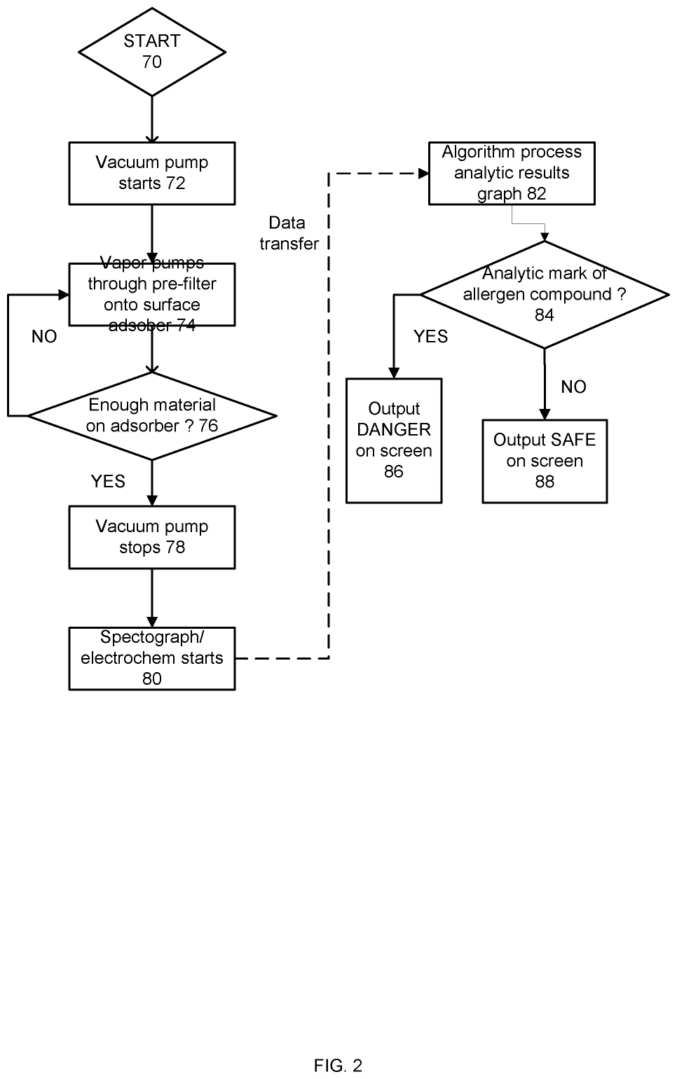

FIG. 2 there is illustrated an operational diagram depicting the various stages in method 71 for operation of the sensor. The sensor may be a hand-held device. The method 71 may START (70). The sensor takes in the vapors coming out of the food. These vapors are then pumped (pump starts 72, the method may check if enough vapor material was aggregated on the filter 76—and if so the pump stops 78—else jump to step 72) or driven through the device, and aggregated (filtered) using a process such as but not limited to Selective Adsorption so only the relevant volatiles are left behind. The filter/adsorber that aggregates the vapors may be preceded by a pre-filter.

The filter may be then then scanned by a spectrograph, and/or an electrochemical potentiostat (step 80) , or any other chemical analysis, and results processed by the sensor and/or are transferred (via USB or Bluetooth or any other communication method) to another computerized entity such as a smartphone. There, an application or another computer program has the data analyzed (step 82) to determine if the allergen is found (step 84), and the results are finally presented (if an allergenic compound is found—display DANGER (step 86)—else present SAFE (step 88). so the user may decide whether the food is safe for them.

There is provided a method of detecting the presence of known food allergens using volatile indicators that indicate the existence of the food which contains the allergens in the dish, by binding said indicators to specific substrate and measuring changes in the substrates electrical characteristics, measurement of its Quartz Crystal Microbalance or scanning it with a spectrometer.

There is provided a method for active detection of allergens in food based on the detection of volatile indicators whose existence in vapors coming out of the dish point to the likelihood of the allergen being present.

For example, peanut allergens such as Ara h 1, 2, 3, 5, 6, and 8 can be detected based on the existence a trace amount of peanut in the dish, which will release to the air volatile compounds and components such as N-methylpyrrole, Isobutylaldehyde, Isobutanoic acid, Arachidic acid or Cyclohexane (for roasted peanuts). These volatiles can be selectively bound to a surface using Molecular Imprinting, Metal-Organic-Framework, or various other surface adsorption methods, and then detected—indicating the existence of the peanut, and hence the peanut allergen, in the food dish.

Testing vapors instead of testing solid samples or fluid samples has the following benefits:
a. Its efficiency is not limited (like solid or liquid based tests) for absolutely homogenous substances.
b. The tested vapors are obtained from most and even all of the food dish, unlike sampling solid or liquid which limits the scope of the analysis to the size of a minimal sample.
c. It is not limited to the detection of antigens—but rather searches for other volatile materials that constitute indicators for the allergen presence.

It is also possible, using this method, to detect any other harmful substances (in food or otherwise) which release specific indicators into the atmosphere around them. For example, meat spoilage releases indicative volatile compound such as indole and cadaverine, which have also been shown to be detectable using this method.

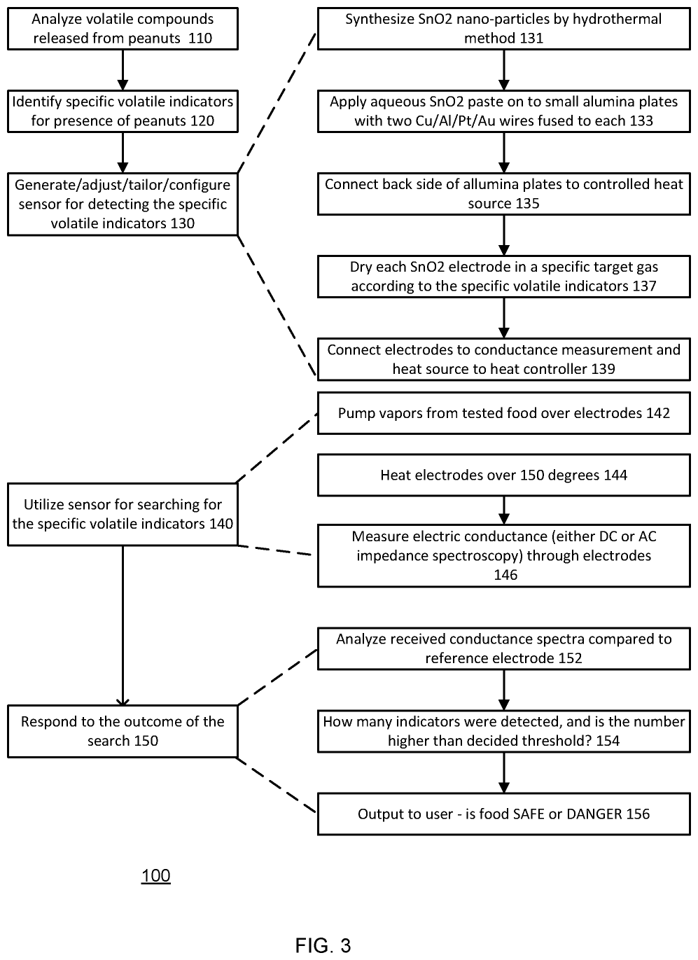

FIG. 3 illustrates a method for providing a sensor and a method for utilizing a sensor. Both methods may be combined—and are collectively denoted 100.

The first method includes a sequence of steps 110, 120 and 130.

Step 110 may include analyzing volatile compounds released from peanuts (raw peanuts and/or roasted peanuts).

Step 120 may include identifying specific volatile indicators for presence of peanuts.

Non-limiting examples of the specific volatile indicators include:
a. 2-Ethyl3,6 Dimethyl Pyrazine—is very abundant in vapors coming from roasted peanuts, and seems to also exist in raw peanuts, in smaller (barely detectable by GCMS-HS) amounts.
b. N-methyl-pyrrole—was found in small amounts, both in raw and roasted peanuts.
c. Coumaran, 2, 3-dihydrobenzofuran—was found in small amounts in peanut vapors, and is very rare in other foodstuffs.

Step 130 may include generating and/or adjusting and/or tailoring and/or configuring a sensor for detecting the specific volatile indicators.

Step 130 may include a sequence of steps 131, 133, 135, 137 and 139. This sequence of steps provide non-limiting examples of a specific method related to a specific sensor.

Step 131 may include synthesizing SnO2 (with or without metal framework) nano-particles by (for example) a hydrothermal method, or other Molecular Imprintable semiconducting nano-materials.

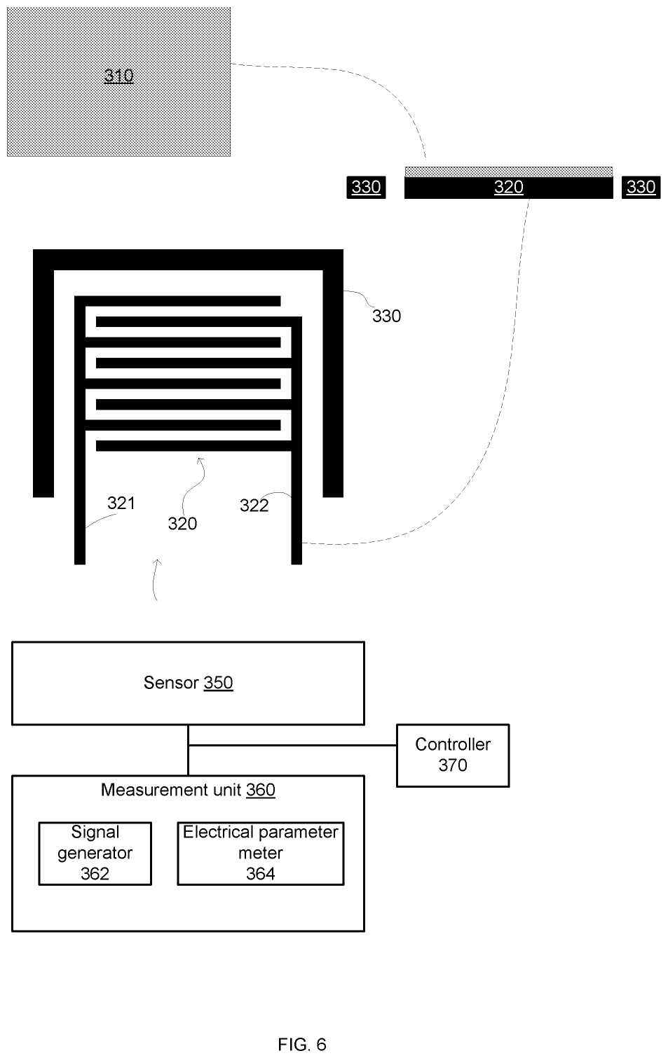

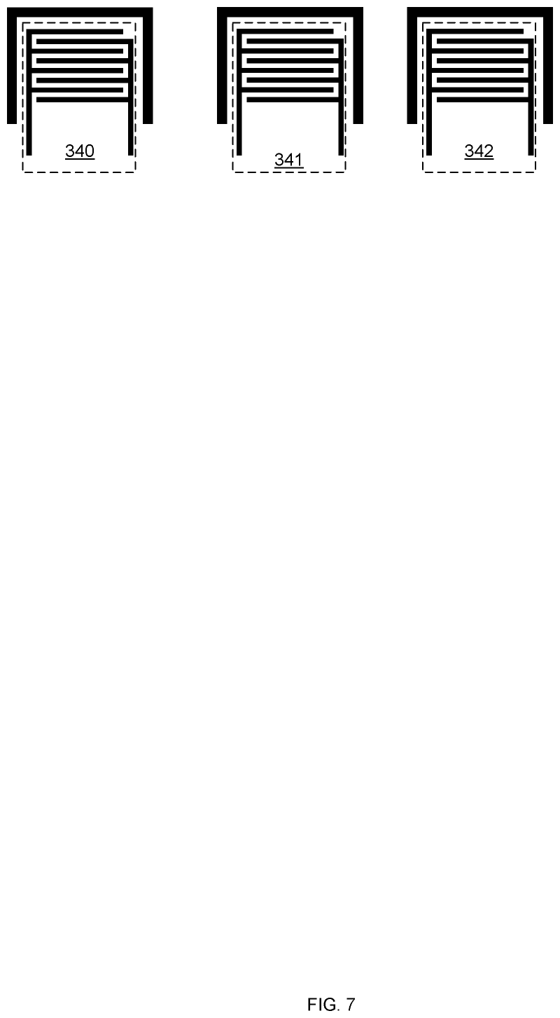

Step 133 may include applying aqueous SnO2 paste on to small alumina (or other ceramic) plates with two Pt/Au/Ag (or any other metal) wires(electrodes) fused to it as described in FIG. 6-7.

Step 135 may include connecting of alumina plates to controlled heat source. It should be noted that it is also possible to have heat source and connection comb fused to different pieces that are in physical contact with each other, and so making the plate with the SnO2 cheaper and expendable.

Step 137 may include drying each SnO2 electrode in a specific target gas according to the specific volatile indicators. Target gas is the specific volatile indicator.

Step 139 may include connecting electrodes to conductance measurement and heating source to heat controller.

The second method includes a sequence of steps 140 and 150.

Step 140 may include a sequence of steps 142, 144 and 146. This sequence of steps provide non-limiting examples of a specific method of activation.

Step 142 may include pumping vapors from tested food over electrodes, or it may include natural intake of said vapors (without active pumping). Step 142 may be replaced by any step (passive or active) of allowing the vapors to reach the electrodes.

Step 144 may include heating electrodes (for example between 120-180 degrees).

Step 146 may include measuring. electric conductance (either DC or AC impedance spectroscopy) through electrodes. It is assumed that molecules of a specific volatile indicator are present in the vapor—they will be attached to the electrode that was tailored to sense the specific volatile indicator (SnO2 electrode dried in the specific volatile indicator)—and this will change the resistance of that electrode. It is especially effective to measure the AC impedance spectroscopy (that is, the change in AC impedance over a varied group of frequencies) to create a special fingerprint to detected indicator. While molecular imprinting does prevent all manner of other molecules from attaching to the filter, there are many other materials with molecular structure which is similar to the required. Adsorption of such related material will also affect a change in conductance, but a scan of impedance over various frequencies will allow the discretion whether or not it is the desired indicator.

Step 150 may include a sequence of steps 152, 154 and 156. This sequence of steps provide non-limiting examples of a specific method of responding to the outcome of a measurement and comparison of the results to a database.

Step 152 may include analyzing the received conductance spectra compared to reference electrode. Analysis may be based on a two-staged machine learning algorithm: the first one based on Decision Tree Classifiers, Neighbors Classifier, Gaussian, SVC or any other classifier that can create rules according to impedance measured per each frequency according to time of exposure; while second stage can be based on Logistic Regression or any other binary-input classifier as to weigh between results derived from each frequency and decide for the existence of required indicator. Other manners of analysis may be provided. The first phase may involve checking, per AC frequency, whether an allergen is found and the second phase may take the outcome of the first phase into account.

Step 154 may include checking how many indicators were detected and is the number higher than decided threshold as to indicate the presence of the source material. Any other checking may be applied. For example—the analysis may not include thresholding.

Step 156 may include determining whether the food is safe or not—and generating an alert indicative of the determination. For example—if there are three specific volatile indicators—and a majority of these specific volatile indicators were found—at an amount that exceeds their thresholds—that the food is not safe. The determination may be responsive to the amount of specific volatile indicator detected, and the like.

Any combination of the steps of any of the methods in the specification may be provided.

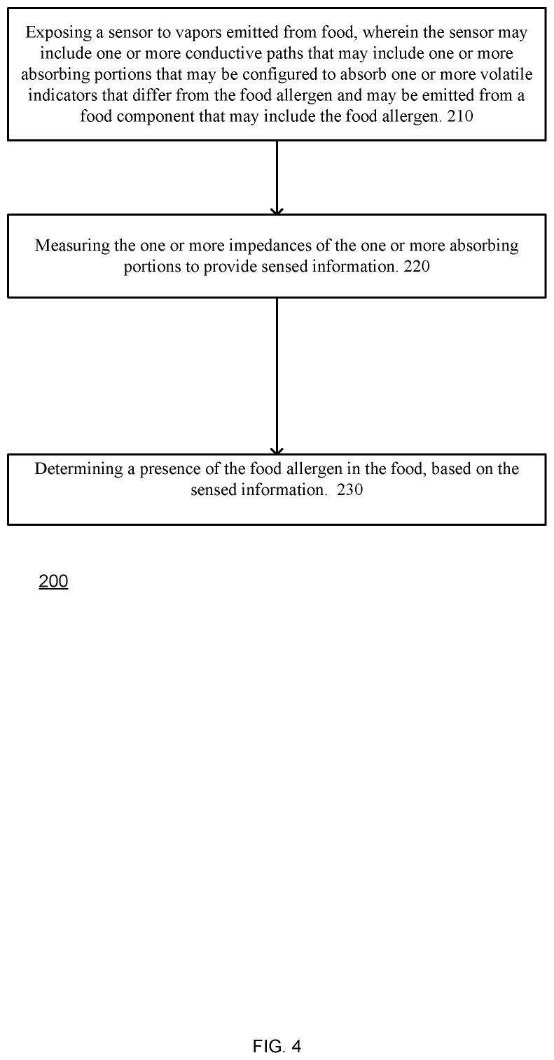

FIG. 4 illustrates method 200 for detecting a food allergen.

Method 200 may include a sequence of step 210, 220, and 230.

Step 210 may include exposing a sensor to vapors emitted from food, wherein the sensor may include one or more conductive paths that may include one or more adsorbing portions that may be configured to adsorb one or more volatile indicators that differ from the food allergen and may be emitted from a food component that may include the food allergen.

The electric impedance of the one or more adsorbing portions may be responsive to the adsorption of at least one volatile indicator of the one or more volatile indicators.

The one or more adsorbing portions may include nanoparticles of response-driven resistance.

The exposing may include positioning the sensor at a location that is expected to receive the vapors, may include actively or passively directing the vapors towards the sensor, and the like.

The different conductive paths of the one or more conductive paths may be tailored to sense different volatile indicators of the one or more volatile indicators.

The one or more adsorbing portions belong to a disposable portion of the mobile handheld device. Thus, the adsorbing portions may be replaced after searching for food allergen in one food item. The disposable portion may be detachably coupled to other parts of the food allergen detector. It may be detached by any movement or manner—sliding movement, pushing, pulling, rotating, and the like.

The exposing of the sensor may include directing the gas flow towards the sensor.

Step 220 may include measuring the one or more impedances of the one or more adsorbing portions to provide sensed information.

The measuring the one or more impedances of the one or more adsorbing portions may include heating the one or more conductive paths.

The measuring of an impedance of an adsorbing portion of the one or more conductive paths may include performing alternating current (AC) impedance spectroscopy.

Thus, the measuring may include multiple iterations of (a) provide an AC signal of a certain frequency to the conductive path, (b) measure the impedance of the adsorbing portion, (c) change the frequency and jump to step (a). There may be any number of iterations—for example few tens of iterations.

Additionally or alternatively, the measuring of the impedance may include measuring the direct current (DC) resistance of the one or more the adsorbing portions.

The sensed information may represent measurements of one or more impedances of the one or more adsorbing portions at different alternating current (AC) frequencies.

Step 230 may include determining a presence of the food allergen in the food, based on the sensed information.

Each volatile indicator may have a signature—for example an AC impedance spectrum. The AC impedance spectrums of the volatile indicators may be compared to the sensed information—in order to detect the presence of the volatile indicator.

The determining of the presence of the allergen may include comparing the sensed information to reference information about impedances of one or more conductive paths at a presence of the one or more volatile indicators at the different AC frequencies.

The method may include general indication of concentration of the allergen in the food based on the sensed information.

Each conductive path may include a pair of electrodes. The adsorbing portion of the one or more adsorbing portions electrically couples the pair of electrodes to each other.

Method 200 may be executed by a mobile handheld device. The mobile handheld device may be compact—for example may weigh less than 2 kilos and have a centimetric scale dimensions.

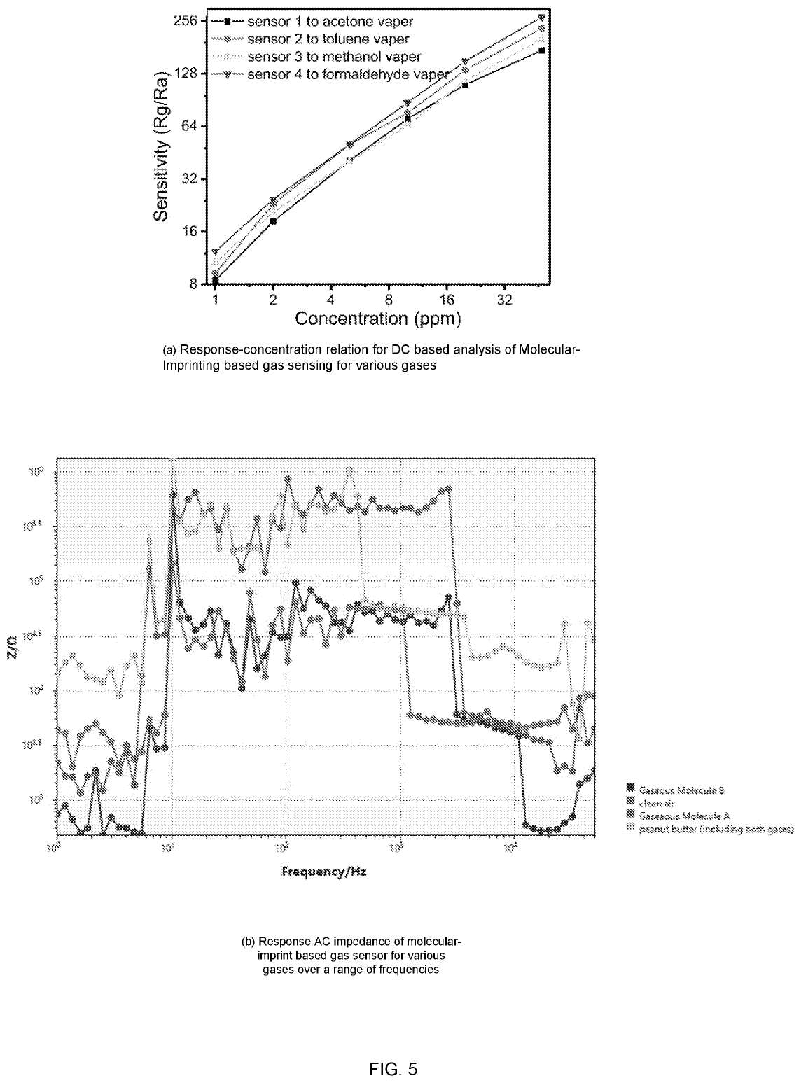

FIG. 5 illustrates the performances of various measurement methods. The top graph (a) depicts DC responses (given by relative resistance change) of similarly-designed Molecular-Imprint based gas sensor, to various gases at various concentrations. The bottom graph (b) depicts AC impedances of a conductive path as described before which was imprinted with a specific gaseous material, denoted as “Gaseous Molecule A”, and exposed to 4 different environments, all with significantly less gas concentrations than the PPM-level in graph (a). In green it is seen what the AC impedance is when exposed to an environment rich in gas A (that is, same gas that was imprinted). In red it is seen what the AC impedance is when exposed to clean air. In blue is the AC impedance when exposed to Gaseous Molecule B—a different, though quite similar in molecular morphology (and hence partially able to connect to the molecular imprints in the nanoparticles), gaseous material. Finally in yellow is the impedance when the conductive path in exposed to gases naturally exhausted from commercial peanut butter, one of which is gas A (though at small quantities). As can be seen, in the frequency range between 5 and 400 Hz the results of the peanut butter and gas A are quite similar, and different from the other options.;

FIG. 6 is an example of a food allergen detector 300 that include a sensor 350, a measurement unit 360, and a controller 370 for controlling the food allergen detector.

The measurement unit 360 may include a signal generator 362 and a meter—such as a current meter or voltage meter 364. The signal generator may generate signals that are fed to the sensor. The signals may be AC signals that in different iterations have different frequencies.

Sensor 350 includes one or more conductive paths. FIG. 6 illustrates a conductive path that includes two electrodes 321 and 322 (collectively denoted 32) on which there is an adsorbing portion 310 (of nanoparticles of response-driven resistance) that electrically couples electrode 321 to electrode 322. FIG. 6 also illustrates a heating element 330 that may heat the vicinity of the electrodes during, before or during the measurements. The heating may evaporate condensed water or other liquid contaminations.

The measurement unit feeds signals to the conductive path and measures the impedance of the conductive path (or rather the impedance of the adsorbing portion).

The measurement unit 360 is configured to (a) measure, while the sensor is exposed to vapors from the food, the one or more impedances of the one or more adsorbing portions to provide sensed information; and (b) to determine a presence of the food allergen in the food, based on the sensed information.

The adsorbing portion is configured to adsorb one or more volatile indicators that differ from the food allergen and are emitted from a food component that comprises the food allergen. An impedance of the adsorbing portion is responsive to an adsorption of at least one volatile indicator of the one or more volatile indicators.

The electrodes may be located in a disposable portion of the food allergen detector—thus allowing the user to replace the (preferably cheap) disposable electrodes between measurements.

FIG. 7 illustrates that the sensor may include multiple conductive pairs—that are located on disposable substrates 340, 341 and 342—wherein different electrodes/conductive paths may be tailored to detect different volatile indicators.

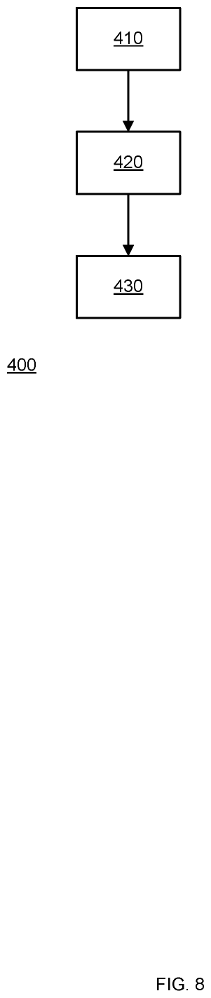

FIG. 8 illustrates method 400.

Method 400 is for detecting an occurrence of an event.

Method 400 may include a sequence of steps 410, 420 and 430.

Step 410 of exposing a sensor to vapors, wherein the sensor comprises one or more conductive paths that comprise one or more adsorbing portions that are configured to adsorb one or more volatile indicators that are indicative of the occurrence of the event; wherein an impedance of the one or more adsorbing portions is responsive to an adsorbance of at least one volatile indicator of the one or more volatile indicators.

Step 420 of measuring the one or more impedances of the one or more adsorbing portions to provide sensed information; wherein the measuring comprises performing alternating current (AC) impedance spectroscopy.

Step 430 of determining the occurrence of an event, based on the sensed information.

The event may be a meat spoilage or any other event.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

The connections as discussed herein may be any type of connection suitable to transfer signals from or to the respective nodes, units or devices, for example via intermediate devices. Accordingly, unless implied or stated otherwise, the connections may for example be direct connections or indirect connections. The connections may be illustrated or described in reference to being a single connection, a plurality of connections, unidirectional connections, or bidirectional connections. However, different embodiments may vary the implementation of the connections. For example, separate unidirectional connections may be used rather than bidirectional connections and vice versa. Also, plurality of connections may be replaced with a single connection that transfers multiple signals serially or in a time multiplexed manner Likewise, single connections carrying multiple signals may be separated out into various different connections carrying subsets of these signals. Therefore, many options exist for transferring signals.

Although specific conductivity types or polarity of potentials have been described in the examples, it will be appreciated that conductivity types and polarities of potentials may be reversed.

Each signal described herein may be designed as positive or negative logic. In the case of a negative logic signal, the signal is active low where the logically true state corresponds to a logic level zero. In the case of a positive logic signal, the signal is active high where the logically true state corresponds to a logic level one. Note that any of the signals described herein may be designed as either negative or positive logic signals. Therefore, in alternate embodiments, those signals described as positive logic signals may be implemented as negative logic signals, and those signals described as negative logic signals may be implemented as positive logic signals.

Furthermore, the terms “assert” or “set” and “negate” (or “deassert” or “clear”) are used herein when referring to the rendering of a signal, status bit, or similar apparatus into its logically true or logically false state, respectively. If the logically true state is a logic level one, the logically false state is a logic level zero. And if the logically true state is a logic level zero, the logically false state is a logic level one.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. A method for detecting a food allergen, the method comprises:
exposing a sensor to vapors emitted from food, wherein the sensor comprises one or more conductive paths that comprise one or more adsorbing portions that are configured to adsorb one or more volatile indicators that differ from the food allergen and are emitted from a food component that comprises the food allergen; wherein an impedance of the one or more adsorbing portions is responsive to an adsorption of at least one volatile indicator of the one or more volatile indicators;
measuring the one or more impedances of the one or more adsorbing portions to provide sensed information; and
determining a presence of the food allergen in the food, based on the sensed information.

2. The method according to claim 1 wherein the measuring of an impedance of an adsorbing portion of the one or more conductive paths comprises performing alternating current (AC) impedance spectroscopy.

3. The method according to claim 1 wherein the sensed information represents measurements of one or more impedances of the one or more adsorbing portions at different alternating current (AC) frequencies, and wherein the determining of the presence of the allergen comprises comparing the sensed information to reference information about impedances of one or more conductive paths at a presence of the one or more volatile indicators at the different AC frequencies.

4. The method according to claim 3 wherein the different AC frequencies do not exceed few tens of frequencies.

5. The method according to claim 1 comprising determining a concentration of the allergen in the food based on the sensed information.

6. The method according to claim 1 wherein each conductive path comprises a pair of electrodes, and wherein an adsorbing portion of the one or more adsorbing portions electrically couples the pair of electrodes to each other.

7. The method according to claim 1 wherein the directing of the vapors, the measuring of the resistance and the determining of the presence of the allergen are executed by a mobile handheld device.

8. The method according to claim 1 wherein different conductive paths of the one or more conductive paths are tailored to sense different volatile indicators of the one or more volatile indicators.

9. The method according to claim 1 wherein the measuring the one or more impedances of the one or more adsorbing portions comprises heating the one or more conductive paths.

10. The method according to claim 1 wherein the exposing of the sensor comprises directing the gas flow towards the sensor.

11. The method according to claim 1 wherein at least one adsorbing portion comprises SnO2 nanoparticles.

12. The method according to claim 1 wherein the one or more volatile indicators comprise 2-Ethyl3,6 Dimethyl Pyrazine.

13. The method according to claim 1 wherein the one or more volatile indicators comprise N-methyl-pyrrole.

14. The method according to claim 1 comprising Coumaran, 2, 3-dihydrobenzofuran.

15. The method according to claim 1 wherein the one or more adsorbing portions belong to a disposable portion of the mobile handheld device.

16. The method according to claim 1 wherein the one or more adsorbing portions comprise nanoparticles of response-driven resistance.

17. A food allergen detector, comprising:
a sensor that comprises one or more conductive paths, wherein the conductive paths comprise one or more adsorbing portions that are configured to adsorb one or more volatile indicators that differ from the food allergen and are emitted from a food component that comprises the food allergen; wherein an impedance of the one or more adsorbing portions is responsive to an adsorption of at least one volatile indicator of the one or more volatile indicators; and
a measurement unit that is configured to (a) measure, while the sensor is exposed to vapors from the food, the one or more impedances of the one or more adsorbing portions to provide sensed information; and (b) to determine a presence of the food allergen in the food, based on the sensed information.

18. The food allergen detector according to claim 17 wherein the measurement unit is configured to measure an impedance of a conductive path of the one or more conductive paths by performing alternating current (AC) impedance spectroscopy.

19. The food allergen detector according to claim 17 wherein the sensed information represents measurements of one or more impedances of the one or more adsorbing portions at different alternating current (AC) frequencies, and wherein the measurement unit is configured to determining the presence of the allergen by comparing the sensed information to reference information about impedances of one or more conductive paths at a presence of the one or more volatile indicators at the different AC frequencies.

20. The food allergen detector according to claim 20 wherein the different AC frequencies do not exceed few tens of frequencies.

21. The food allergen detector according to claim 17 wherein the measurement unit is configured to determine a concentration of the allergen in the food based on the sensed information.

22. The food allergen detector according to claim 17 wherein each conductive path comprises a pair of electrodes, and wherein an adsorbing portion of the one or more adsorbing portions electrically couples the pair of electrodes to each other.

23. The food allergen detector according to claim 17 wherein the food allergen detector is a mobile handheld device.

24. The food allergen detector according to claim 17 wherein different conductive paths of the one or more conductive paths are tailored to sense different volatile indicators.

25. The food allergen detector according to claim 17 comprising one or more heating elements that are configured to heat the one or more conductive paths while the measurement unit measures the one or more impedances of the one or more conductive paths.

26. The food allergen detector according to claim 17 comprising a vapors manipulator for directing the vapors towards the sensor .

27. The food allergen detector according to claim 17 wherein at least one adsorbing portion comprises SnO2 nanoparticles.

28. The food allergen detector according to claim 17 wherein the one or more volatile indicators comprise 2-Ethyl3,6 Dimethyl Pyrazine.

29. The food allergen detector according to claim 17 wherein the one or more volatile indicators comprise N-methyl-pyrrole.

30. The food allergen detector according to claim 17 comprising Coumaran

31. The food allergen detector according to claim 30 wherein the adsorbing portion belongs to a disposable portion of the mobile handheld device.

32. The food allergen detector according to claim 17 wherein the adsorbing portion comprises nanoparticles of variable resistance.

33. The food allergen detector according to claim 17 wherein the food allergen detector is coupled to a mobile handheld device.

34. A method for detecting an occurrence of an event, the method comprises
exposing a sensor to vapors, wherein the sensor comprises one or more conductive paths that comprise one or more adsorbing portions that are configured to adsorb one or more volatile indicators that are indicative of the occurrence of the event; wherein an impedance of the one or more adsorbing portions is responsive to an adsorption of at least one volatile indicator of the one or more volatile indicators;
measuring the one or more impedances of the one or more adsorbing portions to provide sensed information; wherein the measuring comprises performing alternating current (AC) impedance spectroscopy; and
determining the occurrence of an event, based on the sensed information.

35. The method according to claim 34 wherein the event is a meat spoilage.

36. A detector that comprises: a sensor that comprises one or more conductive paths, wherein the conductive paths comprise one or more adsorbing portions that are configured to adsorb one or more volatile indicators that are indicative of an occurrence of an event; wherein the electric impedance of the one or more adsorbing portions is responsive to adsorption of at least one volatile indicator of the one or more volatile indicators; and a measurement unit that is configured to:
(a) measure, while the sensor may be exposed to vapors, the one or more impedances of the one or more adsorbing portions to provide sensed information; wherein the measuring comprises performing alternating current (AC) impedance spectroscopy; and
(b) determine the occurrence of the event, based on the sensed information.

37. The detector according to claim 36 wherein the event is a meat spoilage.
